(19) United States
(12) Patent Application Publication
(10) Pub. No.: US 2023/0048170 A1
(43) Pub. Date: Feb. 16, 2023

(54) COMPOSITION INCLUDING CATIONIC POLYSACCHARIDE AND ANIONIC ANTIOXIDANT

(71) Applicant: L'Oreal, Paris (FR)

(72) Inventors: Tatsushi ISOJIMA, Kawasaki-shi, Kanagawa (JP); Toshifumi SHIROYA, Kawasaki-shi, Kanagawa (JP); Cindy CHHUON, Kawasaki-shi, Kanagawa (JP); Kazuhiko MARUYAMA, Kawasaki-shi, Kanagawa (JP)

(73) Assignee: L'Oreal, Paris (FR)

(21) Appl. No.: 17/785,028

(22) PCT Filed: Dec. 4, 2020
(86) PCT No.: PCT/JP2020/046187

§ 371 Date: Jun. 13, 2022

(30) Foreign Application Priority Data
Dec. 16, 2019 (JP) 2019-226589

(51) Int. Cl. A61K 8/60; A61K 8/81; A61K 8/73; A61K 8/06; A61Q 19/08; A61K 8/02

(52) U.S. Cl. CPC A61K 8/602; A61K 8/817; A61K 8/732; A61K 8/062; A61Q 19/08; A61K 8/0212; A61K 2800/522

(57) ABSTRACT

The present invention relates to a composition comprising: (a) at least one cationic polysaccharide; (b) at least one antioxidant; and (c) water, wherein the (b) antioxidant has at least one negatively chargeable and/or negatively charged moiety selected from the group consisting of a sulfuric group, a sulfate group, a sulfonic group, a sulfonate group, a phosphoric group, a phosphate group, a phosphonic group, a phosphonate group, a phenolic hydroxyl group, a carboxylic group, and a carboxylate group. The composition according to the present invention can include a relatively larger amount of antioxidant.

### TECHNICAL FIELD

The present invention relates to a composition including a combination of a cationic polysaccharide and an anionic antioxidant.

### BACKGROUND ART

The formation of free radicals is a widely accepted pivotal mechanism leading to skin aging. Free radicals are highly reactive molecules with unpaired electrons that can directly damage various cellular membranes, lipids, proteins, RNA and DNA. The damaging effects of these reactive oxygen species are induced internally during normal metabolism and externally through various oxidative stresses. UV exposure and environmental pollution can accelerate skin aging by producing free radicals in skin.

Antioxidants protect cells from the damage of oxidative stress by scavenging free radicals and inhibiting following oxidation reactions. The topical application of antioxidants is broadly used in skin care product to prevent skin aging.

### DISCLOSURE OF INVENTION

However, some antioxidants have limited solubility in water which is often used as a vehicle in topical formulations. Therefore, there has been a need to increase the amount of such less water-soluble antioxidants in a composition including water. Even for antioxidants which is soluble in water, there has also been a need to increase furthermore the amount of such more water-soluble antioxidants.

Thus, the objective of the present invention is to provide a composition which can include a relatively larger amount of antioxidant.

The above objective of the present invention can be achieved by a composition comprising:
(a) at least one cationic polysaccharide;
(b) at least one antioxidant; and
(c) water,
   wherein
   the (b) antioxidant has at least one negatively chargeable and/or negatively charged moiety selected from the group consisting of a sulfuric group, a sulfate group, a sulfonic group, a sulfonate group, a phosphoric group, a phosphate group, a phosphonic group, a phosphonate group, a phenolic hydroxyl group, a carboxylic group, and a carboxylate group.

It is preferable that the (a) cationic polysaccharide and the (b) antioxidant be capable of forming a complex.

The (a) cationic polysaccharide may have at least one quaternary ammonium group.

The (a) cationic polysaccharide may be selected from cationic cellulose polymers, cationic starches, cationic gums and mixtures thereof.

The (a) cationic polysaccharide may be selected from the group consisting of polyquaternium-4, polyquaternium-10, polyquaternium-24, polyquaternium-67, starch hydroxypropyl trimonium chloride, cassia hydroxypropyltrimonium chloride, chitosan, and mixtures thereof.

The amount of the (a) cationic polysaccharide(s) in the composition according to the present invention may be from 0.01% to 10% by weight, preferably from 0.05% to 5% by weight, and more preferably from 0.1% to 2% by weight, relative to the total weight of the composition.

The (b) antioxidant may be sparingly soluble in water.

The (b) antioxidant may be selected from flavonoids, flavonoid glycosides, flavanones, flavanone glycosides, cinnamic acid derivatives, and mixtures thereof.

The (b) antioxidant may be selected from the group consisting of baicalin, rutin, disodium rutinyl disulfate, ferulic acid, and mixtures thereof.

The amount of the (b) antioxidant in the composition according to the present invention may be from 0.05% to 10% by weight, preferably from 0.1% to 5% by weight, and more preferably from 0.15% to 1% by weight, relative to the total weight of the composition.

The amount of the (c) water in the composition according to the present invention may be from 50% to 99% by weight, preferably from 60% to 98% by weight, and more preferably from 70% to 97% by weight, relative to the total weight of the composition.

The composition according to the present invention may further comprise at least one oil and/or at least one organic UV filter. This composition may be in the form of an emulsion.

The composition according to the present invention may be a cosmetic composition, preferably a skin cosmetic composition, and more preferably a skin care cosmetic composition.

The present invention also relates to a cosmetic process for a keratin substance such as skin, comprising:

applying to the keratin substance the composition according to the present invention; and drying the composition to form a cosmetic film on the keratin substance.

### BRIEF DESCRIPTION OF DRAWINGS

FIG. 1A is a graph showing the change in the b* value before and after UV exposure of the membrane surface on which the composition according to Example 4, Comparative Example 2 or Comparative Example 4 has been applied and dried up. FIG. 1B is a graph showing the change in the b* value before and after UV exposure of the membrane surface on which the composition according to Example 5, Comparative Example 3 or Comparative Example 4 has been applied and dried up.

FIG. 2 is a graph showing the change in the b* value before and after UV exposure, in combination with smoke, of the membrane surface on which the composition according to Example 5, Comparative Example 3 or Comparative Example 4 has been applied and dried up.

FIG. 3 is a graph showing the change in the lipid hydroperoxide concentration depending on UV exposure time, in combination with smoke, of the membrane surface on which the composition according to Example 5, Example 6, Example 7 or Comparative Example 3 has been applied and dried up.

FIG. 4A shows a photograph of a glass surface coated with the composition according to Example 7. FIG. 4B shows a photograph of a glass surface coated with the composition according to Comparative Example 5.

### BEST MODE FOR CARRYING OUT THE INVENTION

After diligent research, the inventors have discovered that it is possible to provide a composition which can include a relatively larger amount of antioxidant. Thus, the composition according to the present invention comprises:
(a) at least one cationic polysaccharide;
(b) at least one antioxidant; and
(c) water,
   wherein
   the (b) antioxidant has at least one negatively chargeable and/or negatively charged moiety selected from the group consisting of a sulfuric group, a sulfate group, a sulfonic group, a sulfonate group, a phosphoric group, a phosphate group, a phosphonic group, a phosphonate group, a phenolic hydroxyl group, a carboxylic group, and a carboxylate group.

The composition according to the present invention can include a relatively larger amount of antioxidant than a composition including the antioxidant without the (a) cationic polysaccharide. Thus, the composition according to the present invention is preferable for topical applications such as cosmetic applications.

The (b) antioxidant is anionic or is capable of becoming anionic. Therefore, in the (c) water, the (a) cationic polysaccharide can form a complex with the (b) antioxidant. The formation of the complex can be based on ionic bonds or ionic interactions. Thus, the complex may be a polyelectrolyte complex.

Since the composition according to the present invention can include a relatively larger amount of antioxidant, the composition can exert enhanced anti-oxidation effects.

Furthermore, the (a) cationic polysaccharide can form a film which is useful to uniformly distribute the (b) antioxidant on a substrate, preferably a keratin substance, and more preferably skin. Therefore, the (b) antioxidant can effectively exert anti-oxidation effects on the substrate. The anti-oxidation effects include preventing or reducing oxidation which may be enhanced by UV irradiation and/or environmental pollutions.

In addition, a combination of the (a) cationic polysaccharide and the (b) antioxidant can function to emulsify oil(s). Thus, the composition according to the present invention can include oil or organic UV filter (this is oily), and can be in the form of an emulsion. Also, the (a) cationic polysaccharide can function to reduce the adhesion of pollutants onto a substrate, preferably a keratin substance, and more preferably skin, even if the composition according to the present invention includes oil(s) or organic UV filter(s).

Hereinafter, the composition, process and the like according to the present invention will be explained in a more detailed manner.

[Cationic Polysaccharide]

The composition according to the present invention includes (a) at least one cationic polysaccharide. Two or more different types of (a) cationic polysaccharides may be used in combination. Thus, a single type of (a) cationic polysaccharide or a combination of different types of (a) cationic polysaccharides may be used.

The (a) cationic polysaccharide has a positive charge density. The charge density of the (a) cationic polysaccharide may be from 0.01 meq/g to 20 meq/g, preferably from 0.05 to 15 meq/g, and more preferably from 0.1 to 10 meq/g.

It may be preferable that the molecular weight of the (a) cationic polysaccharide be 500 or more, preferably 1,000 or more, more preferably 2,000 or more, and even more preferably 5,000 or more.

Unless otherwise defined in the description, “molecular weight” may mean a number average molecular weight.

The (a) cationic polysaccharide may have at least one positively chargeable and/or positively charged moiety selected from the group consisting of a primary, a secondary or tertiary amino group, a quaternary ammonium group, a guanidine group, a biguanide group, an imidazole group, an imino group, and a pyridyl group. The term (primary) “amino group” here means the group —NH₂.

It is preferable that the (a) cationic polysaccharide have at least one quaternary ammonium group, preferably a quaternary trialkyl ammonium group, and more preferably a quaternary trimethyl ammonium group.

The quaternary ammonium group may be present in a quaternary ammonium group-containing group which may be represented by the following chemical formula (I):

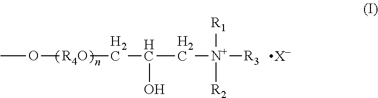

wherein
each of R₁ and R₂ denotes a C₁₋₃ alkyl group, preferably a methyl or ethyl group, and more preferably a methyl group,
R₃ denotes a C₁₋₂₄ alkyl group, preferably a methyl or ethyl group, and more preferably methyl group,
X— denotes an anion, preferably a halide, and more preferably a chloride,
n denotes an integer from 0-30, preferably 0-10, and more preferably 0, and
R₄ denotes a C₁₋₄ alkylene group, preferably an ethylene or propylene group.

The leftmost ether bond (—O—) in the above chemical formula (I) can attach to the sugar ring of the polysaccharide.

It is preferable that the quaternary ammonium group-containing group be —O—CH₂—CH(OH)—CH₂—N⁺(CH₃)₃.

The (a) cationic polysaccharide may be a homopolymer or a copolymer. The term “copolymer” is understood to mean both copolymers obtained from two kinds of monomers and those obtained from more than two kinds of monomers, such as terpolymers obtained from three kinds of monomers.

The (a) cationic polysaccharide may be selected from natural and synthetic cationic polysaccharides.

It may be preferable that the (a) cationic polysaccharide be selected from cationic cellulose polymers. Non-limiting examples of the cationic cellulose polymers are as follows.

(1) Cationic cellulose polymers such as cellulose ether derivatives comprising one or more quaternary ammonium groups described, for example, in French Patent No. 1 492 597, such as the polymers sold under the names “JR” (JR 400, JR 125, JR 30M) or “LR” (LR 400, LR 30M) by the company Dow Chemical. These polymers are also defined in the CTFA dictionary as quaternary ammoniums of hydroxyethylcellulose that have reacted with an epoxide substituted with a trimethylammonium group.

(2) Cationic cellulose polymers such as cellulose copolymers and cellulose derivatives grafted with at least one water-soluble monomer of quaternary ammonium, and described, for example, in U.S. Pat. No. 4,131,576, such as hydroxyalkylcelluloses, for instance, hydroxymethyl-, hydroxyethyl-, and hydroxypropylcelluloses grafted, for example, with at least one chosen from methacryloylethyltrimethylammonium, methacrylamidopropyltrimethylammonium, and dimethyldiallylammonium. Commercial products corresponding to these polymers include, for example, the products sold under the names “Celquat® L 200” and “Celquat® H 100” by the company Akzo Novel.

(3) Cationic cellulose polymers having at least one quaternary ammonium group comprising at least one fatty chain, such as alkyl, arylalkyl or alkylaryl groups comprising at least 8 carbon atoms. It may be preferable that the cationic cellulose polymers be quaternized hydroxyethylcelluloses modified with at least one quaternary ammonium group comprising at least one fatty chain, such as alkyl, arylalkyl or alkylaryl groups comprising at least 8 carbon atoms, or mixtures thereof. The alkyl radicals borne by the quaternary ammonium group may preferably contain from 8 to 30 carbon atoms, especially from 10 to 30 carbon atoms. The aryl radicals preferably denote phenyl, benzyl, naphthyl or anthryl groups. More preferably, the cationic cellulose polymer may comprise at least one quaternary ammonium group including at least one C₈-C₃₀ hydrocarbon group. Examples of quaternized alkylhydroxyethylcelluloses containing C₈-C₃₀ fatty chains that may be mentioned include the products Quatrisoft LM 200, Quatrisoft LM-X 529-18-A, Quatrisoft LM-X 529-18B (C₁₂ alkyl) and Quatrisoft LM-X 529-8 (C₁₈ alkyl) or Softcat Polymer SL100, Softcat SX-1300X, Softcat SX-1300H, Softcat SL-5, Softcat SL-30, Softcat SL-60, Softcat SK-MH, Softcat SX-400X, Softcat SX-400H, SoftCat SK-L, Softcat SK-M, and Softcat SK-H, sold by the company Dow Chemical, and the products Crodacel QM, Crodacel, QL (C12 alkyl) and Crodacel QS (C18 alkyl) sold by the company Croda.

It may also be preferable that the (a) cationic polysaccharide be selected from cationic starches.

As examples of the cationic starches, mention may be made of starches modified with a 2,3-epoxypropyltrimethylammonium salt (e.g. chloride), such as the product known as starch hydroxypropyltrimonium chloride according to the INCl nomenclature and sold under the name SENSOMER C1-50 from Ondeo or Pencare™ DP 1015 from Ingredion.

It may also be preferable that the (a) cationic polysaccharide be selected from cationic gums.

The gums may be, for example, selected from the group consisting of cassia gum, karaya gum, konjac gum, gum tragacanth, tara gum, acacia gum and gum arabic.

Examples of cationic gum include cationic polygalactomannan derivatives such as guar gum derivatives and cassia gum derivatives, e.g., CTFA: Guar Hydroxypropyltrimonium Chloride, Hydroxypropyl Guar Hydroxypropyltrimonium Chloride, and Cassia Hydroxypropyltrimonium Chloride. Guar hydroxypropyltrimonium chloride is commercially available under the Jaguar™ trade name series from Rhodia Inc. and the N-Hance trade name series from Ashland Inc. Cassia Hydroxypropyltrimonium Chloride is commercially available under the Sensomer™ CT-250 and Sensomer™ CT-400 trademarks from Lubrizol Advanced Materials, Inc or the ClearHance™ from Ashland Inc.

It may also be preferable that the (a) cationic polysaccharide be selected from chitosans.

It may be preferable that the (a) cationic polysaccharide be selected from the group consisting of polyquaternium-4, polyquaternium-10, polyquaternium-24, polyquaternium-67, starch hydroxypropyl trimonium chloride, cassia hydroxypropyltrimonium chloride, chitosan, and a mixture thereof.

The amount of the (a) cationic polysaccharide(s) in the composition according to the present invention may be 0.01% by weight or more, preferably 0.05% by weight or more, and more preferably 0.1% by weight or more, relative to the total weight of the composition.

The amount of the (a) cationic polysaccharide(s) in the composition according to the present invention may be 10% by weight or less, preferably 5% by weight or less, and more preferably 2% by weight or less, relative to the total weight of the composition.

The amount of the (a) cationic polysaccharide(s) in the composition according to the present invention may be from 0.01% to 10% by weight, preferably from 0.05% to 5% by weight, and more preferably from 0.1% to 2% by weight, relative to the total weight of the composition.

[Antioxidant]

The composition according to the present invention includes (b) at least one antioxidant. Two or more different types of (b) antioxidants may be used in combination. Thus, a single type of (b) antioxidant or a combination of different types of (b) antioxidants may be used.

The (b) antioxidant has at least one negatively chargeable and/or negatively charged moiety selected from the group consisting of a sulfuric group, a sulfate group, a sulfonic group, a sulfonate group, a phosphoric group, a phosphate group, a phosphonic group, a phosphonate group, a phenolic hydroxyl group, a carboxylic group, and a carboxylate group.

The (b) antioxidant may be sparingly soluble in water.

The term “sparingly water-soluble” here means poor solubility in water. Therefore, a sparingly water-soluble antioxidant used herein is difficult to dissolve in water. Thus, the “sparingly water-soluble” antioxidant may be a compound having a water-solubility less than 1% by weight, preferably less than 0.1% by weight, more preferably less than 0.01% by weight at room temperature (20-25° C., preferably 25° C.) at pH 7 without the (a) cationic polysaccharide. In other words, the “sparingly water-soluble” antioxidant may have a solubility in pure water of less than 1% by weight, preferably less than 0.1% by weight, more preferably less than 0.01% by weight at room temperature (20-25° C., preferably 25° C.) at pH 7.

The (b) antioxidant which may be used in the present invention may be a sparingly water-soluble phenolic compound also having at least one phenolic hydroxyl group in one molecule, or a sparingly water-soluble polyphenol compound also having two or more phenolic hydroxyl groups in one molecule.

As examples of the (b) antioxidant with at least one phenolic hydroxyl group, mention may be made of mangiferin, polydatin, curcumin, and reseveratol.

It is preferable that the (b) antioxidant be not ellagic acid.

The (b) antioxidant which can be used in the present invention may be selected from flavonoids and non-flavonoids.

The flavonoids may be selected from a group consisting of chalcones, flavones such as luteolin, baicalein and diosmetin, flavanones such as hesperetin, flavanols, flavonols, dihydroflavonols, isoflavonoids, neoflavonoids, catechins, anthocyanidins, tannins, and derivatives thereof.

The (b) antioxidant may be in the form of a salt form or a glycoside form.

The flavonoids may be in the form of flavonoid glycosides. As examples of the flavonoid glycosides, mention may be made of baicalin, rutin, and diosmin.

The flavanones may be in the form of flavanone glycosides. As examples of the flavanone glycosides, mention may be made of glucosyl hesperidin.

The non-flavonoids may be selected from a group consisting of lignans, aurones, curcuminoids, and other phenylpropanoids, and derivatives thereof, such as resorcinol derivatives.

The (b) antioxidant which can be used in the present invention may be selected from cinnamic acid derivatives.

The cinnamic acid derivative may be represented by the following chemical formula (II).

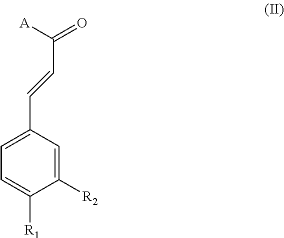

wherein
A is chosen from
an OR₃ group wherein R₃ is chosen from a hydrogen atom, a phytyl group, a benzyl group, a linear or branched C₁-C₁₈ alkyl group, a C₃-C₈ cycloalkyl group, a C₃-C₈ cycloalkyl-C₁-C₅ alkyl group, an alkali metal ion, an alkaline earth metal ion and an ammonium ion,
   and
an NHR₄ group wherein R₄ is chosen from a hydrogen atom, a phytyl group, a benzyl group, and a linear or branched C₁-C₁₈ alkyl group, a C₃-C₈ cycloalkyl group, a C₃-C₈ cycloalkyl-C₁-C₅ alkyl group,
   R₁ is chosen from a hydrogen atom, a hydroxyl group, a C₁-C₆ alkoxy group, a linear or branched C₁-C₁₈ alkyl group, a C₃-C₈ cycloalkyl group, a C₃-C₈ cycloalkyl-C₁-C₅ alkyl group, and
   R₂ is chosen from a hydrogen atom, a hydroxyl group, and a C₁-C₆ alkoxy group;

As the linear or branched C₁-C₁₈ alkyl group, preferably C₁-C₁₂ alkyl group, and more preferably C₁-C₆ alkyl group, mention may be made of, for example, a methyl group, an ethyl group, an n-propyl group, an i-propyl group, an n-butyl group, an i-butyl group, a sec-butyl group, a tert-butyl group, an n-pentyl group, an i-pentyl group, an 1-ethylpropyl group, a hexyl group, an isohexyl group, and an 1-ethylbutyl group.

As the C₃-C₈ cycloalkyl group, mention may be made of, for example, a cyclopropyl group, a cyclobutyl group, a cyclopentyl group, and a cyclohexyl group.

As the C₃-C₈ cycloalkyl-C₁-C₅ alkyl group, mention may be made of, for example, a cyclopropylmethyl group, a cyclobutylmethyl group, a cyclopentylmethyl group, and a cyclohexylmethyl group.

As the C₁-C₆ alkoxy group, mention may be made of, for example, a methoxy group, an ethoxy group, an n-propoxy group, an i-propoxy group, an n-butoxy group, an i-butoxy group, a sec-butoxy group, a tert-butoxy group, an n-pentyoxy group, an i-pentyoxy group, an 1-ethylpropoxy group, a hexyloxy group, an isohexyloxy group, and an 1-ethylbutoxy group. A methoxy group is preferable.

It may be preferable that R₁ be a hydroxyl group, and that R₂ be chosen from a hydroxyl group and a C₁-C₆ alkoxy group, more preferably a methoxy group.

As the cinnamic acid derivative, mention may be made of, for example, 2-ethylhexyl methoxycinnamate, isopropyl methoxycinnamate, isoamyl methoxycinnamate, diisopropyl methoxycinnamate, caffeic acid, ferulic acid. Caffeic acid and ferulic acid may be preferable, and ferulic acid may be more preferable.

The (b) antioxidant may be water-soluble.

The term “water-soluble” here means being soluble in water. Therefore, a water-soluble antioxidant used herein is not difficult to dissolve in water. Thus, the “water-soluble” antioxidant may be a compound having a water-solubility of 1% by weight or more at room temperature (20-25° C., preferably 25° C.) at pH 7 without the (a) cationic polysaccharide. In other words, the “water-soluble” antioxidant may have a solubility in pure water of 1% by weight or more at room temperature (20-25° C., preferably 25° C.) at pH 7.

The (b) antioxidant which may be used in the present invention may be a water-soluble phenolic compound also having at least one phenolic hydroxyl group in one molecule, or a water-soluble polyphenol compound also having two or more phenolic hydroxyl groups in one molecule.

The water-soluble antioxidant which can be used in the present invention may be selected from water-soluble derivatives of flavonoids and non-flavonoids both of which are explained above.

The water-soluble derivatives of flavonoids and non-flavonoids may be selected from sulfates, sulfonates, phosphates, phosphonates, and carboxylates of flavonoids and non-flavonoids.

The water-soluble antioxidant may be selected from sulfates of flavonoids, preferably sulfates of flavonoid glycosides, and more preferably disodium rutinyl disulfate.

It may be preferable that the (b) antioxidant be selected from flavonoids, flavonoid glycosides, flavanones, flavanone glycosides, cinnamic acid derivatives, and mixtures thereof.

It may be more preferable that the (b) antioxidant be selected from the group consisting of baicalin, rutin, disodium rutinyl disulfate, ferulic acid, and mixtures thereof.

The amount of the (b) antioxidant(s) in the composition according to the present invention may be 0.05% by weight or more, preferably 0.1% by weight or more, and more preferably 0.15% by weight or more, relative to the total weight of the composition.

The amount of the (b) antioxidant(s) in the composition according to the present invention may be 10% by weight or less, preferably 5% by weight or less, and more preferably 1% by weight or less, relative to the total weight of the composition.

The amount of the (b) antioxidant(s) in the composition according to the present invention may be from 0.05% to 10% by weight, preferably from 0.1% to 5% by weight, and more preferably from 0.15% to 1% by weight, relative to the total weight of the composition.

[Water]

The composition according to the present invention includes (c) water.

The amount of the (c) water may be 50% by weight or more, preferably 60% by weight or more, and more preferably 70% by weight or more, relative to the total weight of the composition.

The amount of the (c) water may be 99% by weight or less, preferably 98% by weight or less, and more preferably 97% by weight or less, relative to the total weight of the composition.

The amount of the (c) water may be from 50 to 99% by weight, preferably from 60 to 98% by weight, and more preferably from 70 to 97% by weight, relative to the total weight of the composition.

[pH]

The pH of the composition according to the present invention may be from 3 to 9, preferably from 3.5 to 8.5, and more preferably from 4 to 8.

At a pH of from 3 to 9, the composition according to the present invention can be very stable.

The pH of the composition according to the present invention may be adjusted by adding at least one alkaline agent and/or at least one acid. The pH of the composition according to the present invention may also be adjusted by adding at least one buffering agent.

(Alkaline Agent)

The composition according to the present invention may comprise at least one alkaline agent. Two or more alkaline agents may be used in combination. Thus, a single type of alkaline agent or a combination of different types of alkaline agents may be used.

The alkaline agent may be an inorganic alkaline agent. It is preferable that the inorganic alkaline agent be selected from the group consisting of ammonia; alkaline metal hydroxides; alkaline earth metal hydroxides; alkaline metal phosphates and monohydrogenophosphates such as sodium phosphate or sodium monohydrogen phosphate.

As examples of the inorganic alkaline metal hydroxides, mention may be made of sodium hydroxide and potassium hydroxide. As examples of the alkaline earth metal hydroxides, mention may be made of calcium hydroxide and magnesium hydroxide. As an inorganic alkaline agent, sodium hydroxide is preferable.

The alkaline agent may be an organic alkaline agent. It is preferable that the organic alkaline agent be selected from the group consisting of monoamines and derivatives thereof; diamines and derivatives thereof; polyamines and derivatives thereof; basic amino acids and derivatives thereof; oligomers of basic amino acids and derivatives thereof; polymers of basic amino acids and derivatives thereof; urea and derivatives thereof; and guanidine and derivatives thereof.

As examples of the organic alkaline agents, mention may be made of alkanolamines such as mono-, di- and tri-ethanolamine, and isopropanolamine; urea, guanidine and their derivatives; basic amino acids such as lysine, ornithine or arginine; and diamines such as those described in the structure below:

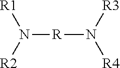

wherein R denotes an alkylene such as propylene optionally substituted by a hydroxyl or a C₁-C₄ alkyl radical, and R₁, R₂, R₃ and R₄ independently denote a hydrogen atom, an alkyl radical or a C₁-C₄ hydroxyalkyl radical, which may be exemplified by 1,3-propanediamine and derivatives thereof. Arginine, urea and monoethanolamine are preferable.

The alkaline agent(s) may be used in a total amount of from 0.01% to 15% by weight, preferably from 0.05% to 10% by weight, more preferably from 0.1% to 5% by weight, relative to the total weight of the composition, depending on their solubility.

(Acid)

The composition according to the present invention may comprise at least one acid. Two or more acids may be used in combination. Thus, a single type of acid or a combination of different types of acids may be used.

As the acid, mention may be made of any inorganic or organic acids, preferably inorganic acids, which are commonly used in cosmetic products. A monovalent acid and/or a polyvalent acid may be used. A monovalent acid such as citric acid, lactic acid, sulfuric acid, phosphoric acid and hydrochloric acid (HCl) may be used. HCl is preferable.

The acid(s) may be used in a total amount of from 0.01% to 15% by weight, preferably from 0.05% to 10% by weight, more preferably from 0.1% to 5% by weight, relative to the total weight of the composition, depending on their solubility.

(Buffering Agent)

The composition according to the present invention may comprise at least one buffering agent. Two or more buffering agents may be used in combination. Thus, a single type of buffering agent or a combination of different types of buffering agents may be used.

As the buffering agent, mention may be made of an acetate buffer (for example, acetic acid+sodium acetate), a phosphate buffer (for example, sodium dihydrogen phosphate+di-sodium hydrogen phosphate), a citrate buffer (for example, citric acid+sodium citrate), a borate buffer (for example, boric acid+sodium borate), a tartrate buffer (for example, tartaric acid+sodium tartrate dihydrate), Tris buffer (for example, tris(hydroxymethyl)aminomethane), Hepes buffer (4-(2-hydroxyethyl)-1-piperazineethanesulfonic acid).

[Oil]

The composition according to the present invention may include at least one oil. If two or more oils are used, they may be the same or different.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The oil may be a non-polar oil such as a hydrocarbon oil, a silicone oil, or the like; a polar oil such as a plant or animal oil and an ester oil or an ether oil; or a mixture thereof.

The oil may be selected from the group consisting of oils of plant or animal origin, synthetic oils, silicone oils, hydrocarbon oils and fatty alcohols.

As examples of plant oils, mention may be made of, for example, apricot oil, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols, and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters, as well as pentaerythrityl tetraethyl hexanoate.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrityl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate) and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used according to the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:

(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of formula:

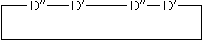

with D″:

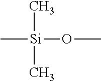

and with D′:

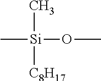

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and

(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

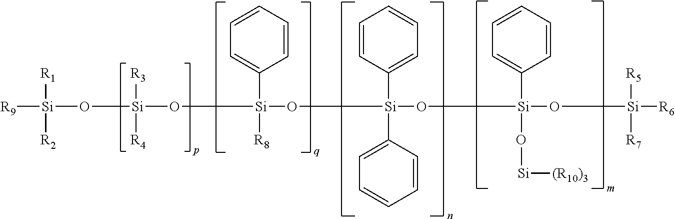

in which
R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl or butyl radicals, and
m, n, p and q are, independently of each other, integers 0 to 900 inclusive, preferably 0 to 500 inclusive, and more preferably 0 to 100 inclusive,
with the proviso that the sum n+m+q is other than 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250 and SF 1265.

As the phenyl silicone oil, phenyl trimethicone (R₁ to R₁₀ are methyl; p, q, and n=0; m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that the fatty alcohol be a saturated fatty alcohol.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

According to at least one embodiment, the fatty alcohol used in the composition according to the present invention is preferably chosen from octyldodecanol, hexyldecanol and mixtures thereof.

The amount of the oil(s) in the composition according to the present invention may be 0.01% by weight or more, preferably 0.05% by weight or more, and more preferably 0.1% by weight or more, relative to the total weight of the composition.

The amount of the oil(s) in the composition according to the present invention may be 50% by weight or less, preferably 40% by weight or less, and more preferably 30% by weight or less, relative to the total weight of the composition.

The amount of the oil(s) in the composition according to the present invention may be from 0.01% to 50% by weight, preferably from 0.05% to 40% by weight, and more preferably from 0.1% to 30% by weight, relative to the total weight of the composition.

[Organic UV Filter]

The composition according to the present invention may include at least one organic UV filter. If two or more organic UV filters are used, they may be the same or different, preferably the same.

The organic UV filter used for the present invention may be active in the UV-A and/or UV-B region. The organic UV filter may be hydrophilic and/or lipophilic.

The organic UV filter may be solid or liquid. The terms “solid” and “liquid” mean solid and liquid, respectively, at 25° C. under 1 atm.

The organic UV filter can be selected from the group consisting of anthranilic compounds; dibenzoylmethane compounds; cinnamic compounds; salicylic compounds; camphor compounds; benzophenone compounds; β,β-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from α-alkylstyrene; 4,4-diarylbutadiene compounds; guaiazulene and derivatives thereof; rutin and derivatives thereof; and mixtures thereof.

Mention may be made, as examples of the organic UV filter(s), of those denoted below under their INCI names, and mixtures thereof.
Anthranilic compounds: Menthyl anthranilate, marketed under the trademark “Neo Heliopan MA” by Haarmann and Reimer.
Dibenzoylmethane compounds: Butyl methoxydibenzoylmethane, marketed in particular under the trademark “Parsol 1789” by Hoffmann-La Roche; and isopropyl dibenzoylmethane.
Cinnamic compounds: Ethylhexyl methoxycinnamate, marketed in particular under the trademark “Parsol MCX” by Hoffmann-La Roche; isopropyl methoxycinnamate; isopropoxy methoxycinnamate; isoamyl methoxycinnamate, marketed under the trademark “Neo Heliopan E 1000” by Haarmann and Reimer; cinoxate (2-ethoxyethyl-4-methoxy cinnamate); DEA methoxycinnamate; diisopropyl methylcinnamate; and glyceryl ethylhexanoate dimethoxycinnamate.
Salicylic compounds: Homosalate (homomentyl salicylate), marketed under the trademark “Eusolex HMS” by Rona/EM Industries; ethylhexyl salicylate, marketed under the trademark “Neo Heliopan OS” by Haarmann and Reimer; glycol salicylate; butyloctyl salicylate; phenyl salicylate; dipropyleneglycol salicylate, marketed under the trademark “Dipsal” by Scher; and TEA salicylate, marketed under the trademark “Neo Heliopan TS” by Haarmann and Reimer.
Camphor compounds, in particular, benzylidenecamphor derivatives: 3-benzylidene camphor, manufactured under the trademark “Mexoryl SD” by Chimex; 4-methylbenzylidene camphor, marketed under the trademark “Eusolex 6300” by Merck; benzylidene camphor sulfonic acid, manufactured under the trademark “Mexoryl SL” by Chimex; camphor benzalkonium methosulfate, manufactured under the trademark “Mexoryl SO” by Chimex; terephthalylidene dicamphor sulfonic acid, manufactured under the trademark “Mexoryl SX” by Chimex; and polyacrylamidomethyl benzylidene camphor, manufactured under the trademark “Mexoryl SW” by Chimex.
Benzophenone compounds: Benzophenone-1 (2,4-dihydroxybenzophenone), marketed under the trademark “Uvinul 400” by BASF; benzophenone-2 (Tetrahydroxybenzophenone), marketed under the trademark “Uvinul D50” by BASF; Benzophenone-3 (2-hydroxy-4-methoxybenzophenone) or oxybenzone, marketed under the trademark “Uvinul M40” by BASF; benzophenone-4 (hydroxymethoxy benzophonene sulfonic acid), marketed under the trademark “Uvinul MS40” by BASF; benzophenone-5 (Sodium hydroxymethoxy benzophenone Sulfonate); benzophenone-6 (dihydroxy dimethoxy benzophenone); marketed under the trademark “Helisorb 11” by Norquay; benzophenone-8, marketed under the trademark “Spectra-Sorb UV-24” by American Cyanamid; benzophenone-9 (Disodium dihydroxy dimethoxy benzophenonedisulfonate), marketed under the trademark “Uvinul DS-49” by BASF; benzophenone-12, and n-hexyl 2-(4-diethylamino-2-hydroxybenzoyl)benzoate (UVINUL A+ by BASF).
β,β-Diphenylacrylate compounds: Octocrylene, marketed in particular under the trademark “Uvinul N539” by BASF; and Etocrylene, marketed in particular under the trademark “Uvinul N35” by BASF.
Triazine compounds: Diethylhexyl butamido triazone, marketed under the trademark “Uvasorb HEB” by Sigma 3V; 2,4,6-tris(dineopentyl 4′-aminobenzalmalonate)-s-triazine, bis-ethylhexyloxyphenol methoxyphenyl triazine marketed under the trademark «TINOSORB S» by CIBA GEIGY, and ethylhexyl triazone marketed under the trademark «UVINUL T150» by BASF.
Benzotriazole compounds, in particular, phenylbenzotriazole derivatives: 2-(2H-benzotriazole-2-yl)-6-dodecyl-4-methylpheno, branched and linear; and those described in U.S. Pat. No. 5,240,975.
Benzalmalonate compounds: Dineopentyl 4′-methoxybenzalmalonate, and polyorganosiloxane comprising benzalmalonate functional groups, such as polysilicone-15, marketed under the trademark “Parsol SLX” by Hoffmann-LaRoche.
Benzimidazole compounds, in particular, phenylbenzimidazole derivatives: Phenylbenzimidazole sulfonic acid, marketed in particular under the trademark “Eusolex 232” by Merck, and disodium phenyl dibenzimidazole tetrasulfonate, marketed under the trademark “Neo Heliopan AP” by Haarmann and Reimer.
Imidazoline compounds: Ethylhexyl dimethoxybenzylidene dioxoimidazoline propionate.
Bis-benzoazolyl compounds: The derivatives as described in EP-669,323 and U.S. Pat. No. 2,463,264.
Para-aminobenzoic acid compounds: PABA (p-aminobenzoic acid), ethyl PABA, Ethyl dihydroxypropyl PABA, pentyl dimethyl PABA, ethylhexyl dimethyl PABA, marketed in particular under the trademark “Escalol 507” by ISP, glyceryl PABA, and PEG-25 PABA, marketed under the trademark “Uvinul P25” by BASF.
Methylene bis-(hydroxyphenylbenzotriazol) compounds, such as 2,2′-methylenebis[6-(2H-benzotriazol-2-yl)-4-methyl-phenol] marketed in the solid form under the trademark “Mixxim BB/200” by Fairmount Chemical, 2,2′-methylenebis[6-(2H-benzotriazol-2-yl)-4-(1,1,3,3-tetramethylbutyl)phenol] marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184 and EP-893,119, and
   Drometrizole trisiloxane, marketed under the trademark “Silatrizole” by Rhodia Chimie or “Mexoryl XL” by L'Oreal, as represented below.

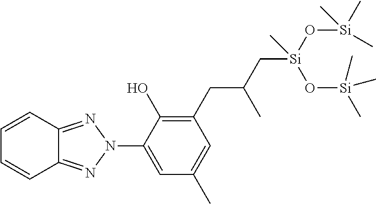 [SMILES: Cc1cc(CC(C)C[Si](C)(O[Si](C)(C)C)O[Si](C)(C)C)c(O)c(-n2nc3ccccc3n2)c1]

Benzoxazole compounds: 2,4-bis[5-1 (dimethylpropyl)benzoxazol-2-yl-(4-phenyl)imino]-6-(2-ethylhexyl)imino-1,3,5-triazine, marketed under the trademark Uvasorb K2A by Sigma 3V.
Screening polymers and screening silicones: The silicones described in WO 93/04665.
Dimers derived from α-alkylstyrene: The dimers described in DE-19855649.
4,4-Diarylbutadiene compounds: 1,1-dicarboxy(2,2′-dimethylpropyl)-4,4-diphenylbutadiene.

It is preferable that the organic UV filter(s) be selected from the group consisting of:

butyl methoxydibenzoylmethane, ethylhexyl methoxycinnamate, homosalate, ethylhexyl salicylate, octocrylene, phenylbenzimidazole sulfonic acid, benzophenone-3, benzophenone-4, benzophenone-5, n-hexyl 2-(4-diethylamino-2-hydroxybenzoyl)benzoate, piperazinediyl)bis[1-[2-[4-(diethylamino)-2-hydroxybenzoyl]phenyl]-methanone 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone, 2,4,6-tris(dineopentyl 4′-aminobenzalmalonate)-s-triazine, 2,4,6-tris(diisobutyl 4′-aminobenzalmalonate)-s-triazine, 2,4-bis-(n-butyl 4′-aminobenzalmalonate)-6-[(3-{1,3,3,3-tetramethyl-1-[(trimethylsilyloxy]-disiloxanyl}propyl)amino]-s-triazine, 2,4,6-tris-(di-phenyl)-triazine, 2,4,6-tris-(ter-phenyl)-triazine, methylene bis-benzotriazolyl tetramethylbutylphenol, drometrizole trisiloxane, polysilicone-15, dineopentyl 4′-methoxybenzalmalonate, 1,1-dicarboxy(2,2′-dimethylpropyl)-4,4-diphenylbutadiene, 2,4-bis[5-1 (dimethylpropyl)benzoxazol-2-yl-(4-phenyl)imino]-6-(2-ethylhexyl)imino-1,3,5-triazine, camphor benzylkonium methosulfate and mixtures thereof.

The amount of the organic UV filter(s) in the composition according to the present invention may be 0.01% by weight or more, preferably 0.05% by weight or more, and more preferably 0.1% by weight or more, relative to the total weight of the composition.

The amount of the organic UV filter(s) in the composition according to the present invention may be 50% by weight or less, preferably 40% by weight or less, and more preferably 30% by weight or less, relative to the total weight of the composition.

The amount of the organic UV filter(s) in the composition according to the present invention may be from 0.01% to 30% by weight, preferably from 0.05% to 20% by weight, and more preferably from 0.1% to 10% by weight, relative to the total weight of the composition.

[Optional Additives]

The composition according to the present invention may comprise, in addition to the aforementioned components, components typically employed in cosmetics, specifically, surfactants or emulsifiers, hydrophilic or lipophilic thickeners, organic volatile or non-volatile solvents, silicones and silicone derivatives other than the oil, natural extracts derived from animals or vegetables, waxes, and the like, within a range which does not impair the effects of the present invention.

The composition according to the present invention may comprise the above optional additive(s) in an amount of from 0.01% to 50% by weight, preferably from 0.05% to 30% by weight, and more preferably from 0.1% to 10% by weight, relative to the total weight of the composition.

The composition according to the present invention may include at least one surfactant or emulsifier. The surfactant or emulsifier may be selected from the group consisting of anionic surfactants, amphoteric surfactants, cationic surfactants, and nonionic surfactants. Two or more surfactants may be used in combination. Thus, a single type of surfactant or a combination of different types of surfactants may be used.

However, it is preferable that the composition according to the present invention include a very limited amount of surfactant(s) or emulsifier(s). The amount of the surfactant(s) or emulsifier(s) in the composition according to the present invention may be 1% by weight or less, preferably 0.1% by weight or less, and more preferably 0.01% by weight or less, relative to the total weight of the composition. It is in particular preferable that the composition according to the present invention include no surfactant or emulsifier.

[Composition]

The composition according to the present invention may be intended to be used as a cosmetic composition. Thus, the cosmetic composition according to the present invention may be intended for application onto a keratin substance. Keratin substance here means a material containing keratin as a main constituent element, and examples thereof include the skin, scalp, nails, lips, hair, and the like. Thus, it is preferable that the cosmetic composition according to the present invention be used for a cosmetic process for the keratin substance, in particular skin.

Thus, the cosmetic composition according to the present invention may be a skin cosmetic composition, preferably a skin care composition or a skin makeup composition, in particular a composition for protecting skin from UV light and/or pollutants in the air.

The composition according to the present invention may be in any form such as a solution, a dispersion, an emulsion, a gel, and a paste. If the composition according to the present invention includes at least one oil and/or at least one organic UV filter, the composition according to the present invention may be in the form of an emulsion such as W/O, O/W, W/O/W and O/W/O, preferably, an O/W emulsion.

The composition according to the present invention can be prepared by mixing the above essential and optional ingredients in accordance with any of the processes which are well known to those skilled in the art.

[Film]

The composition according to the present invention can be used for easily preparing a film.

Thus, the present invention may also relate to a process for preparing a film, preferably a cosmetic film, optionally with a thickness of, preferably more than 0.1 μm, more preferably 1.5 μm or more, and even more preferably 2 μm or more, comprising:

applying onto a substrate, preferably a keratin substance, more preferably skin, the composition according to the present invention; and
drying the composition.

The upper limit of the thickness of the film prepared by the process according to the present invention is not limited. Thus, for example, the thickness of the film may be 1 mm or less, preferably 500 μm or less, more preferably 300 μm or less, and even more preferably 100 μm or less.

Since the process for preparing a film according to the present invention includes the steps of applying the composition according to the present invention onto a substrate, preferably a keratin substance, and more preferably skin, and of drying the composition, the process according to the present invention does not need any spin coating or spraying, and therefore, it is possible to easily prepare even a relatively thick film. Thus, the process for preparing a film according to present invention can prepare a relatively thick film without any special equipment such as spin coaters and spraying machines.

Even if the film prepared by the process according to the present invention is relatively thick, it is still thin and may be transparent, and therefore, may not be easy to perceive. Thus, the film prepared by the process according to the present invention can be used preferably as a cosmetic film.

If the substrate is not a keratin substance such as skin, the composition according to the present invention may be applied onto a substrate made from any material other than keratin. The materials of the non-keratinous substrate are not limited. Two or more materials may be used in combination. Thus, a single type of material or a combination of different types of materials may be used. In any event, it is preferable that the substrate be flexible or elastic.

If the substrate is not a keratin substance, it is preferable that the substrate be water-soluble, because it is possible to leave the film prepared by the process according to the present invention by washing the substrate with water. As examples of the water-soluble materials, mention may be made of poly(meth) acrylic acids, polyethyleneglycols, polyacrylamides, polyvinylalcohol (PVA), starch, celluloseacetates, and the like. PVA is preferable.

If the non-keratinous substrate is in the form of a sheet, it may have a thickness of more than that of the film prepared by the process according to the present invention, in order to ease the handling of the film attached to the substrate sheet. The thickness of the non-keratinous substrate sheet is not limited, but may be from 1 μm to 5 mm, preferably from 10 μm to 1 mm, and more preferably from 50 to 500 μm.

It is more preferable that the film prepared by the process according to the present invention be releasable from the non-keratinous substrate. The mode of release is not limited. Therefore, the film prepared by the process according to the present invention may be peeled from the non-keratinous substrate, or released by the dissolution of the substrate sheet into a solvent such as water.

The present invention may also relate to:
(1) A film, preferably a cosmetic film, optionally with a thickness of, preferably more than 0.1 μm, more preferably 1.5 μm or more, and even more preferably 2 μm or more, prepared by a process comprising:
   applying onto a substrate, preferably a keratin substance, and more preferably skin, the composition according to the present invention; and
   drying the composition,
      and
(2) A film, preferably a cosmetic film, optionally with a thickness of, preferably more than 0.1 μm, more preferably 1.5 μm or more, and even more preferably 2 μm or more, comprising:
   at least one cationic polysaccharide;
   at least one antioxidant; and
   optionally at least one oil,
   wherein
   the antioxidant has at least one negatively chargeable and/or negatively charged moiety selected from the group consisting of a sulfuric group, a sulfate group, a sulfonic group, a sulfonate group, a phosphoric group, a phosphate group, a phosphonic group, a phosphonate group, a phenolic hydroxyl group, a carboxylic group, and a carboxylate group.

The above explanations regarding the cationic polysaccharide and the antioxidant as well as the above oil can apply to those in the above film (1) and (2).

The film thus obtained above can be self-standing. The term “self-standing” here means that the film can be in the form of a sheet and can be handled as an independent sheet without the assistance of a substrate or support. Thus, the term “self-standing” may have the same meaning as “self-supporting”.

It is preferable that the film according to the present invention be hydrophobic.

The term “hydrophobic” in the present specification means that the solubility of the polymer in water (preferably with a volume of 1 liter) at from 20 to 40° C., preferably from 25 to 40° C., and more preferably from 30 to 40° C. is less than 10% by weight, preferably less than 5% by weight, more preferably less than 1% by weight, and even more preferably less than 0.1% by weight, relative to the total weight of the polymer. It is most preferable that the polymer is not soluble in water.

If the film according to the present invention is hydrophobic, the film can have water-resistant properties, and therefore, it can remain on a keratin substance such as skin even if the surface of the keratin substance is wet due to, for example sweat and rain. Thus, when the film according to the present invention provides any cosmetic effect, the cosmetic effect can last a long time.

On the other hand, the film according to the present invention can be easily removed from a keratin substance such as skin under alkaline conditions such as a pH of from 8 to 12, preferably from 9 to 11. Therefore, the film according to the present invention is difficult to remove with water, while it can be easily removed with a soap which can provide such alkaline conditions.

The film according to the present invention may comprise at least one biocompatible and/or biodegradable polymer layer. Two or more biocompatible and/or biodegradable polymers may be used in combination. Thus, a single type of biocompatible and/or biodegradable polymer or a combination of different types of biocompatible and/or biodegradable polymers may be used.

The term “biocompatible” polymer in the present specification means that the polymer does not have excess interaction between the polymer and cells in the living body including the skin, and the polymer is not recognized by the living body as a foreign material.

The term “biodegradable” polymer in the present specification means that the polymer can be degraded or decomposed in a living body due to, for example, the metabolism of the living body itself or the metabolism of the microorganisms which may be present in the living body. Also, the biodegradable polymer can be degraded by hydrolysis.

If the film according to the present invention includes a biocompatible and/or biodegradable polymer, it is less irritable or not irritable to the skin, and does not cause any rash. In addition, due to the use of a biocompatible and/or biodegradable polymer, the cosmetic sheet according to the present invention can adhere well to the skin.

The film according to the present invention can be used for cosmetic treatments of keratin substances, preferably skin, in particular the face. The film according to the present invention can be in any shape or form. For example, it can be used as a full-face mask sheet, or a patch for a part of the face such as the cheek, nose, and around the eyes.

If the film according to the present invention includes at least one hydrophilic or water-soluble UV filter, it can provide UV shielding effects derived from the hydrophilic or water-soluble UV filter. Normally, a hydrophilic or water-soluble UV filter can be removed from the surface of a keratinous substrate such as skin by water such as sweat and rain. However, since the hydrophilic or water-soluble UV filter is included in the film according to the present invention, it is difficult for the hydrophilic or water-soluble UV filter to be removed by water, thereby resulting in long-lasting UV shielding effects.

[Cosmetic Process and Use]

The present invention also relates to:

a cosmetic process for a keratin substance such as skin, comprising: applying to the keratin substance the composition the present invention; and drying the composition to form a cosmetic film on the keratin substance; and
a use of the composition according to the present invention for the preparation of a cosmetic film on a keratin substance such as skin.

The cosmetic process here means a non-therapeutic cosmetic method for caring for and/or making up the surface of a keratin substance such as skin.

In both the above process and use, the above cosmetic film is resistant to water with a pH of 7 or less, and is removable with water with a pH of more than 7, preferably 8 or more, and more preferably 9 or more.

In other words, the above cosmetic film can be water-resistant under neutral or acidic conditions such as a pH of 7 or less, preferably in a range of 6 or more and 7 or less, and more preferably in a range of 5 or more and 7 or less, while the above cosmetic film can be removed under alkaline conditions such as a pH of more than 7, preferably 8 or more, and more preferably 9 or more. The upper limit of the pH is preferably 13, more preferably 12, and even more preferably 11.

Accordingly, the above cosmetic film can be water-resistant, and therefore, it can remain on a keratin substance such as skin even if the surface of the keratin substance is wet due to, for example sweat and rain. On the other hand, the above cosmetic film can be easily removed from a keratin substance such as skin under alkaline conditions. Therefore, the film according to the present invention is difficult to remove with water, while it can be easily removed with a soap which can provide alkaline conditions.

If the above cosmetic film includes a UV filter which may be present in the composition according to the present invention, the above cosmetic film can protect a keratin substance such as skin from UV rays, thereby limiting the darkening of the skin, improving the colour and uniformity of the complexion, and/or treating aging of the skin.

Furthermore, the above cosmetic film can have cosmetic effects such as capturing sebum, matting the appearance of a keratin substrate such as skin, absorbing or adsorbing malodor, and/or protecting the keratin substance from, for example, dirt or pollutant, due to the properties of the film, even if the cosmetic film does not include any cosmetic active ingredient.

In addition, the above cosmetic film may immediately change or modify the appearance of the skin by changing light reflection on the skin and the like, even if the cosmetic film does not include any cosmetic active ingredient. Therefore, it may be possible for the above cosmetic film to conceal skin defects such as pores or wrinkles. Further, the above cosmetic film may immediately change or modify the feel to the touch of the skin by changing the surface roughness on the skin and the like. Furthermore, the above cosmetic film may immediately protect the skin by covering the surface of the skin and shielding the skin, as a barrier, from environmental stresses such as pollutants, contaminants and the like.

The above cosmetic effects can be adjusted or controlled by changing the chemical composition, the thickness and/or the surface roughness of the above cosmetic film.

If the above cosmetic film includes at least one additional cosmetic active ingredient other than the oil and organic UV filter, the cosmetic film can have cosmetic effects provided by the additional cosmetic active ingredient(s). For example, if the cosmetic film includes at least one cosmetic active ingredient selected from anti-aging agents, anti-sebum agents, deodorant agents, anti-perspirant agents, whitening agents and a mixture thereof, the cosmetic film can treat the aging of the skin, absorbing sebum on the skin, controlling odors on the skin, controlling perspiration on the skin, and/or whitening of the skin.

It is also possible to apply a makeup cosmetic composition onto the cosmetic film or sheet according to the present invention after being applied onto the skin.

### EXAMPLES

The present invention will be described in a more detailed manner by way of examples. However, they should not be construed as limiting the scope of the present invention.

### Examples 1-3 and Comparative Example 1

(Preparations)

Compositions according to Examples 1-3 and Comparative Example 1 were prepared by mixing the ingredients shown in Table 1. The numerical values for the amounts of the ingredients shown in Table 1 are all based on “% by weight” as active raw materials.

TABLE 1

| | | | | Comp. |
|---|---|---|---|---|
| | Ex. 1 | Ex. 2 | Ex. 3 | Ex. 1 |
| Polyquaternium-67 | 0.2 | 0.35 | 0.5 | — |
| Baicalin | 0.1 | 0.2 | 0.25 | 0.025 |
| NaOH | qs pH 10 | qs pH 10 | qs pH 10 | qs pH 10 |
| Water | qsp 100 | qsp 100 | qsp 100 | qsp 100 |

In Examples 1-3, a polyquaternium-67 (PQ-67) was dissolved in water to prepare a PQ-67 aqueous solution. Then, the pH of the solution was adjusted to 10 by adding 1M NaOH aqueous solution. Next, baicalin was added to the alkaline solution as much as possible. In the compositions according to Examples 1-3, PQ-67 was able to be solubilized up to an amount of 0.1% by weight, 0.2% by weight and 0.25% by weight, respectively, relative to the total weight of the composition.

On the other hand, in the composition according to Comparative Example 1 including no PQ-67, the solubilized amount of PQ-67 was limited only to 0.025% by weight relative to the total weight of the composition.

The solubility of baicalin was increased in accordance with the increase in the amount of PQ-67, because the positive charges due to PQ-67 increased and the electrostatic interaction between the positive charges of PQ-67 and the negative charges of baicalin also increased. Thus, by forming a complex by the electrostatic interaction, the solubility of baicalin increased up to 10 folds.

Due to the presence of a cationic polymer such as PQ-67, it is possible to increase the solubility of baicalin in water.

The above evaluation shows that the issue of limited solubility of baicalin in water can be solved by forming a polyelectrolyte complex between baicalin and a cationic polymer such as PQ-67.

(Evaluation)

The compositions according to Examples 1-3 and Comparative Example 1 were dropped down onto a substrate and dried up. The compositions according to Examples 1-3 formed a film in which baicalin was uniformly dispersed in the film. On the other hand, the composition according to Comparative Example 1 did not form a film, but formed fine crystals of baicalin at the peripherals of the droplet on the substrate.

Due to a film of a cationic polymer such as PQ-67, it is possible to uniformly distribute an antioxidant such as baicalin on a substrate such as a keratin substance.

### Examples 4-7 and Comparative Examples 2-4

(Preparations)

Compositions according to Examples 4-7 and Comparative Examples 2-4 were prepared by mixing the ingredients shown in Tables 2 and 3. The numerical values for the amounts of the components shown in Tables 2 and 3 are all based on “% by weight” as active raw materials.

TABLE 2

| | | | Comp. | Comp. | Comp. |
|---|---|---|---|---|---|
| | Ex. 4 | Ex. 5 | Ex. 2 | Ex. 3 | Ex. 4 |
| Polyquaternium-67 | 0.5 | 0.5 | — | — | — |
| Baicalin | 0.16 | — | 0.16 | — | — |
| Disodium Rutinyl | — | 0.16 | — | 0.16 | — |
| Disulfate | | | | | |
| NaOH | qs pH 10 | — | qs pH 10 | — | — |
| Water | qsp 100 | qsp 100 | qsp 100 | qsp 100 | qsp 100 |

TABLE 3

| | Ex. 6 | Ex. 7 |
|---|---|---|
| Cassia Hydroxypropyltrimonium Chloride | 0.5 | — |
| Starch Hydroxypropyltrimonium Chloride | — | 0.5 |
| Disodium Rutinyl Disulfate | 0.16 | 0.16 |
| Water | qsp 100 | qsp 100 |

(Evaluation 1)

The effects of a complex of antioxidant (baicalin or disodium rutinyl disulfate) and of a cationic polymer (PQ-67) against oxidative stress were evaluated by using β-carotene bleaching.

UV irradiation is a major source of oxidative stress. Thus, this evaluation was performed by exposing a β-carotene-coated-membrane to UV irradiation, and by tracking the bleaching of the membrane with a spectrocolorimeter.

A membrane was first coated with a β-carotene solution in capric/caprylic triglycerides in an amount of 0.5% by weight relative to the total weight of the solution. As β-carotene is a highly conjugated molecule, it had vivid red/orange color.

Next, 100 mg each of the compositions according to Examples 4-5 and Comparative Examples 2-4 was applied onto the β-carotene-coated membrane, and dried up.

The color of the surface of the membrane was measured with a spectrocolorimeter (CM-3600d by Konica Minolta) in order to determine the b* value based on CIE1976 of the membrane surface.

Then, the membrane surface was exposed to UV light. Due to the action by UV light, β-carotene was oxidized. Thus, the color of β-carotene faded progressively to pale yellow hue. After the exposure to UV light, the color of the membrane surface was measured again in the same manner in order to determine the b* value based on CIE1976 of the membrane surface.

The difference in the b* value before and after the exposure to UV light was determined from the measured b*values.

The oxidation of β-carotene resulted in the change in b* value along the b* axis (blue/yellow axis) in the L*a*b* system under CIE1976.

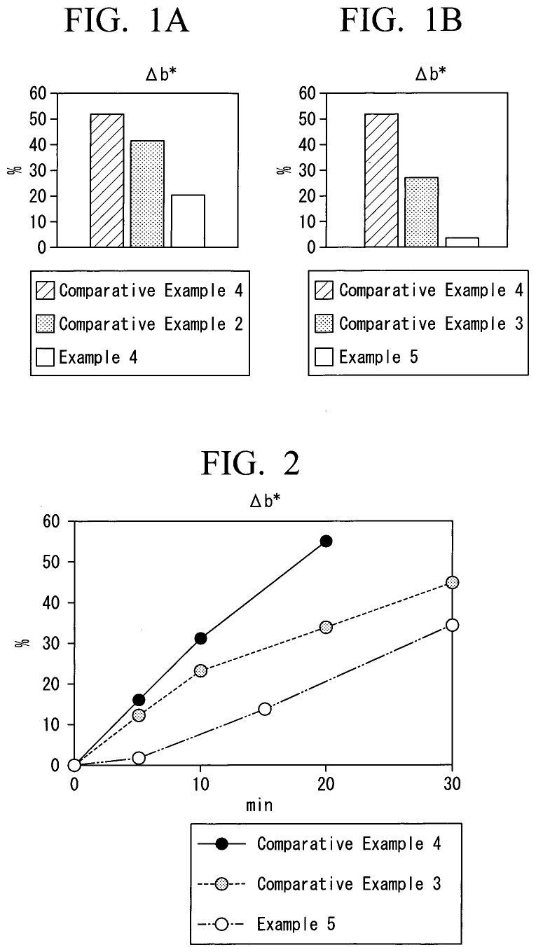

The results of the evaluation are shown in FIG. 1.

As shown in Comparative Example 4, β-carotene was degraded by UV light so that the color of the β-carotene-coated membrane faded

As shown in Comparative Examples 2-3, the anti-oxidation effects by baicalin and disodium rutinyl disulfate reduced the degradation of β-carotene so that the color fading of the 0-carotene-coated membrane was reduced.

As shown in Examples 4-5, the anti-oxidation effects by baicalin and disodium rutinyl disulfate were enhanced by PQ-67 so that the degradation of β-carotene was further reduced and the color fading of the β-carotene-coated membrane was also further reduced.

The above results could be due to a homogeneous distribution of baicalin or disodium rutinyl disulfate in a film formed by PQ-67, which contributed to further reduction of the degradation of β-carotene.

(Evaluation 2)

The effects of a complex of antioxidant (disodium rutinyl disulfate) and of a cationic polymer (PQ-67) against a combination of oxidative stress and air pollution was evaluated by using β-carotene bleaching.

As mentioned above, UV irradiation is a major source of oxidative stress. Furthermore, by being combined with atmospheric pollution exposure, damage caused by oxidative stress can be amplified.

Tobacco smoke, having a similar chemical composition as atmospheric pollution, can trigger biological pathways that make up inflammatory cascade. Thus, it is convenient to use tobacco smoke as a simulation model for atmospheric pollution.

A cellulose membrane was first coated by a β-carotene solution in capric/caprylic triglycerides in an amount of 0.5% by weight relative to the total weight of the solution.

Next, 200 mg each of the compositions according to Example 5 and Comparative Examples 3-4 was applied onto the β-carotene-coated membrane, and dried up.

Each membrane was placed into a hermetic box containing smoke produced by three cigarettes for 30 minutes. Then, each membrane was irradiated by UV light and the change in the b* value in the L*a*b* system under CIE1976 which reflected βcarotene bleaching was tracked by a spectrocolorimeter (CM-3600d by Konica Minolta) at fixed intervals.

The results are shown in FIG. 2.

As being exposed longer to UV irradiation, bleaching of β-carotene was more pronounced as shown in the line for Comparative Example 4 in FIG. 2. Coating the β-carotene-coated membrane with a disodium rutinyl disulfate solution according to the composition according to Comparative Example 3 was able to limit the degradation of β-carotene by 10% to 25% as shown in the line for Comparative Example 3 in FIG. 2. Coating the β-carotene-coated membrane with a disodium rutinyl disulfate/PQ-67 solution according to the composition according to Example 5 was able to limit the decomposition of β-carotene furthermore as shown in the line for Example 5 in FIG. 2.

The above further reduction in the degradation of β-carotene in Example 5 could be provided by a more homogeneous distribution of the antioxidant (disodium rutinyl disulfate) in a film of PQ-67 which can maintain the antioxidant evenly spread onto the membrane.

(Evaluation 3)

The effects of a complex of antioxidant (disodium rutinyl disulfate) and of a variety of cationic polymers against a combination of oxidative stress and air pollution was evaluated in a different manner.

Squalene makes up 15% by weight of human sebum and is its main component. Oxidation of this unsaturated hydrocarbon by tobacco and UV exposure can create lipid hydro-peroxides. The concentration of lipid hydro-peroxides can be measured by redox reaction with ferrous ions that produce ferric ions. These ions can be made to react with thiocyanate ions to induce a color change. Thus, UV-visible spectrophotometry can be appropriate analysis technique to indirectly measure lipid hydro-peroxides concentration.

Squalene hydro-peroxides were prepared by first coating squalene (300 mg) onto two Whatman glassfiber membranes, followed by spreading each (150 mg) of the compositions according to Examples 5-7 and Comparative Example 3 onto each of the membranes. Second, the whole membranes were exposed to tobacco smoke for 30 minutes in a hermetic box containing smoke produced by three cigarettes. Then, the membranes were exposed to UV light for fixed intervals.

After each irradiation interval, an equal size of piece of membrane was cut to extract lipid hydro-peroxides from it by ultra-sonication in chloroform. Then, the lipid hydro-peroxides were made to react with ferrous ions to produce ferric ions. Ferric ions were then quantified by UV-visible spectrophotometry through reaction with thiocyanate ions. A lipid hydro-peroxides assay kit was purchased from Cayman chemical.

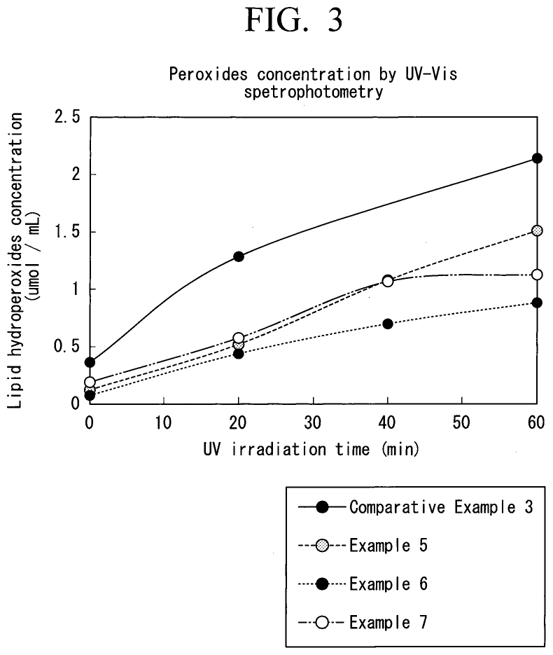

The results are shown in FIG. 3.

Before any UV exposure, the hydro-peroxide concentrations on the membranes for the compositions according to Examples 5-7 was already higher than that on the membrane for the composition according to Comparative Example 3. This could be due to a better or more homogeneous coating of disodium rutinyl disulfate provided by the cationic polymers.

As UV exposure duration became longer, the hydro-peroxide concentrations increased. However, the complex of disodium rutinyl disulfate/cationic polymer provided a better anti-oxidation/anti-pollution protection.

### Examples 7-8 and Comparative Examples 5-6

(Preparations)

Compositions according to Examples 7-8 and Comparative Examples 5-6 were prepared by mixing the components shown in Table 4. The numerical values for the amounts of the components shown in Table 4 are all based on “% by weight” as active raw materials.

TABLE 4

| | | | Comp. | Comp. |
|---|---|---|---|---|
| | Ex. 7 | Ex. 8 | Ex. 5 | Ex. 6 |
| Polyquaternium-67 | 0.5 | — | — | — |
| Starch | — | 0.5 | — | 0.5 |
| Hydroxypropyltrimonium | | | | |
| Chloride | | | | |
| Baicalin | — | 0.16 | — | — |
| Disodium Rutinyl | 0.16 | — | 0.16 | — |
| Disulfate | | | | |
| Diisopropyl Sebacate | 10 | 10 | 10 | 10 |
| Glyceryl Stearate (and) | — | — | 5 | — |
| PEG-100 Stearate | | | | |
| Water | qsp 100 | qsp 100 | qsp 100 | qsp 100 |

(Evaluation 1)

The adhesion of pollution particles, more specifically the role of oil in this phenomenon, was studied through a carbon black deposition test.

On a glass plate, 30 mg each of the compositions according to Example 7 and Comparative Example 5, both of which were in the form of an emulsion, was applied. After drying up the composition in an incubator at 45° C. for 5 minutes, 4 mg of activated charcoal particles as a model of pollution particles were also applied onto the glass plate. An excess amount of activated charcoal particles were then shaken off before incubating the glass plate for 5 minutes again. The surface of the glass plate was then observed with a microscope and the image thereof was further analyzed with the software Image J to determine the percentage of adsorbed activated charcoal particles.

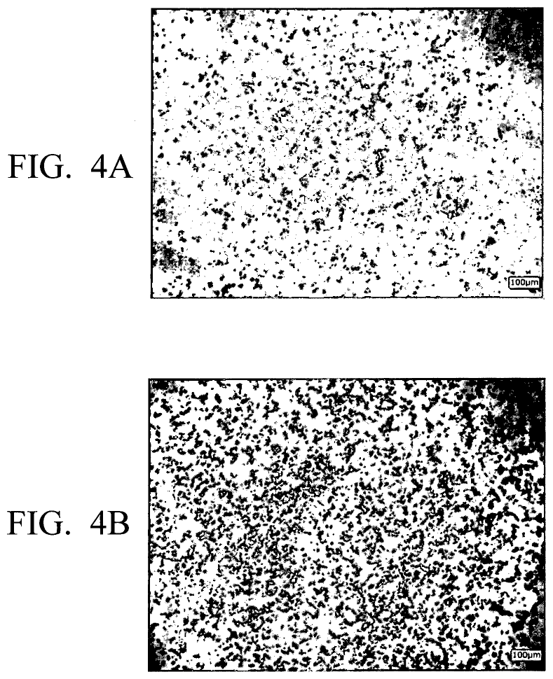

The photographs of the glass surface are shown in FIG. 4. The photograph of FIG. 4A shows the glass surface coated with the composition according to Example 7. The photograph of FIG. 4B shows the glass surface coated with the composition according to Comparative Example 5.

By direct observation, the glass surface which had been coated with the composition according to Example 7 seemed to have a less amount of adsorbed carbon particles than that which had been coated with the composition according to Comparative Example 5.

The results of the image analysis with Image J are as follows.

TABLE 5

| | | Comp. |
|---|---|---|
| | Ex. 7 | Ex. 5 |
| Mean Percentage of Adsorbed Particles | 13.60% | 23.40% |

As oil usually attracts the deposition of pollution particles, the above results demonstrate that a cationic polymer can reduce the deposition of pollution particles on a substrate such as a keratin substance. This could be based on the noteworthy capacity of a complex of the cationic polymer and the antioxidant to trap oil.

(Evaluation 2)

The compositions according to Example 8 and Comparative Example 6 were charged into transparent bottles. The aspect of each bottle was visually observed.

The composition according to Example 8 was in the form of an emulsion such that it was uniform and no phase separation caused.

The composition according to Comparative Example 6 caused phase separation and was not in the form of an emulsion.

The above results demonstrate that a complex composed of the cationic polymer and the antioxidant had emulsification ability. The cation parts of the cationic polymer and the anion parts of the antioxidant made an ion complex which can be hydrophobic parts such that these parts could emulsify oil. On the other hand, without the antioxidant, it was not possible to make an emulsion and the phase separation occurred.

Next, the composition according to Example 8 was also subjected to an observation with a microscope. It was found that the oil droplets are formed, and therefore, the composition was in the form of an O/W emulsion.

Since the cationic polymer and the antioxidant can be of natural origin, the above results imply that emulsions can be prepared with natural materials without artificial chemical materials such as artificial surfactants.

1. A composition, comprising:
(a) at least one cationic polysaccharide;
(b) at least one antioxidant; and
(c) water, wherein
the (b) antioxidant has at least one negatively chargeable and/or negatively charged moiety selected from the group consisting of a sulfuric group, a sulfate group, a sulfonic group, a sulfonate group, a phosphoric group, a phosphate group, a phosphonic group, a phosphonate group, a phenolic hydroxyl group, a carboxylic group, and a carboxylate group.

2. The composition according to claim 1, wherein the (a) cationic polysaccharide and the (b) antioxidant are capable of forming a complex.

3. The composition according to claim 1, wherein the (a) cationic polysaccharide has at least one quaternary ammonium group.

4. The composition according to claim 1, wherein the (a) cationic polysaccharide is selected from cationic cellulose polymers, cationic starches, cationic gums, and mixtures thereof.

5. The composition according to claim 1, wherein the (a) cationic polysaccharide is selected from the group consisting of polyquaternium-4, polyquaternium-10, polyquaternium-24, polyquaternium-67, starch hydroxypropyl trimonium chloride, cassia hydroxypropyltrimonium chloride, chitosan, and mixtures thereof.

6. The composition according to claim 1, wherein the amount of the (a) cationic polysaccharide(s) in the composition is from 0.01% to 10% by weight, relative to the total weight of the composition.

7. The composition according to claim 1, wherein the (b) antioxidant is sparingly soluble in water.

8. The composition according to claim 1, wherein the (b) antioxidant is selected from flavonoids, flavonoid glycosides, flavanones, flavanone glycosides, cinnamic acid derivatives, and mixtures thereof.

9. The composition according to claim 1, wherein the (b) antioxidant is selected from the group consisting of baicalin, rutin, disodium rutinyl disulfate, ferulic acid, and mixtures thereof.

10. The composition according to claim 1, wherein the amount of the (b) antioxidant in the composition is from 0.05% to 10% by weight, relative to the total weight of the composition.

11. The composition according to claim 1, wherein the amount of the (c) water in the composition is from 50% to 99% by weight, relative to the total weight of the composition.

12. The composition according to claim 1, wherein the composition further comprises at least one oil and/or at least one organic UV filter.

13. The composition according to claim 12, wherein the composition is in the form of an emulsion.

14. The composition according to claim 1, wherein the composition is a cosmetic composition.

15. A cosmetic process for a keratin substance such as skin, comprising:
applying to the keratin substance the composition according to claim 1, and
drying the composition to form a cosmetic film on the keratin substance.
